(19) United States
(12) Patent Application Publication
(10) Pub. No.: US 2012/0046513 A1
(43) Pub. Date: Feb. 23, 2012

(54) Magnetic Hot / Cold Pack

(76) Inventor: Michael G. O'Connell, Raynham, MA (US)

(21) Appl. No.: 12/858,144

(22) Filed: Aug. 17, 2010

(51) Int. Cl. A61F 7/02; A61N 2/08

(52) U.S. Cl. 600/15; 607/114; 607/108

(57) ABSTRACT

A pack has a flexible pouch having a pair of planar sheets. The sheets each have a top, a bottom, and two opposite side edges. The sheets are secured together along their top, bottom, and side edges to form a compartment. A gel is carried in the compartment of the pouch. The gel is capable of being cooled and retaining the lack of heat. A plurality of magnets is carried in the compartment of the pouch. In an embodiment, the magnets are permanent magnets. In an embodiment, the permanent magnets are formed of a rare-earth alloy. In an embodiment, the gel is capable of being heated and retaining the heat. In an embodiment, the gel is capable of being cooled and retaining the lack of heat. In an embodiment the gel is a non-toxic biodegradable gel.

### FIELD OF THE INVENTION

The present invention relates to a hot/cold pack and more particularly to a hot/cold pack that has integrated magnets.

### BACKGROUND OF THE INVENTION

Human beings are looking for ways to treat injuries or pain. One such method of treating or therapy is to use cold, an item that draws heat away from the body. Cold is generally used for immediate treatment of acute injuries because it reduces swelling and pain. The cold is a vaso-constrictor because it narrows blood vessels. Cold therapy is also helpful in treating some overuse injuries or chronic pains in athletes.

Another such method of therapy is heat therapy. Heat is generally used for chronic injuries or injuries that have no inflammation or swelling, such as sore or stiff muscles or joint pain. The heat increases circulation and is used stimulate blood flow or increase the elasticity of the muscles.

Another such method is magnetic therapy. One theory of magnetic therapy is that triggers it faster blood flow which results in more oxygen being applied to the specific area of the body because of the faster flow. With more oxygen, the natural healing ability of the person is increased.

Unfortunately conventional systems result in the user having to select only one of the three treatments (i.e., the user can select in the alternative adjusting the temperature either with heat or cold or applying magnetic therapy.)

### SUMMARY

In contrast to the conventional hot and cold therapy gel pack, the pack of the instant invention has the synergy of both temperature and magnetic property. In addition, the pack allows for accurate positioning of the pack related to the area of treatment.

In an embodiment of a pack, the pack has a flexible pouch having a pair of planar sheets. The sheets each have a top, a bottom, and two opposite side edges. The sheets are secured together along their top, bottom, and side edges to form a compartment. A gel is carried in the compartment of the pouch. The gel is capable of being cooled and retaining the lack of heat. A plurality of magnets is carried in the compartment of the pouch.

In an embodiment, the gel is a non-toxic biodegradable gel.

In an embodiment, the magnets are permanent magnets. In an embodiment, the permanent magnets are formed of a rare-earth alloy.

In an embodiment of a pack, the pack has a flexible pouch having a plurality of sheets to form at least one compartment. A gel is carried in the compartment of the pouch. The gel is capable of being adjusted to various temperatures and retaining the non-ambient temperature for a period of time. A plurality of magnets are carried in the compartment of the pouch.

In an embodiment, the gel is capable of being heated and retaining the heat. In an embodiment, the gel is capable of being cooled and retaining the lack of heat. In an embodiment the gel is a non-toxic biodegradable gel.

In a method of therapy of a body, the method includes providing a pack having a flexible pouch including a plurality of sheets to form at least one compartment that carries a gel. The pack is placed against the body such that the plurality of magnets carried in the compartment of the pouch are in proximity to the body to allow for magnetic therapy. The temperature of the body is adjusted in proximity of the pack by the gel pouch.

In a method, the pack including the gel is heated before applying to the body. In a method, the pack including the gel is cooled before applying to the body.

In a method, the pack is designed for and applied to the neck. In another method, the pack is designed for and applied to the lower back. In another method, the pack is designed for running along the spine.

These aspects of the invention are not meant to be exclusive and other features, aspects, and advantages of the present invention will be readily apparent to those of ordinary skill in the art when read in conjunction with the following description, appended claims, and accompanying drawings.

### BRIEF DESCRIPTION OF THE DRAWINGS

A full understanding of the invention can be gained from the following description of its preferred embodiments when read in conjunction with the accompanying drawing in which:

FIG. 1 is a side view of a pack according to the invention;

FIG. 2 is a sectional view of the pack of FIG. 1 taken along the line 2-2;

FIG. 3 is a perspective view of an alternative embodiment of the pack on the neck of a human;

FIG. 4 is a perspective view of an alternative embodiment of a pack on the back of a human; and

FIG. 5 is a front view of an alternative embodiment of a pack for being placed in engagement with the back along the spine.

### PREFERRED EMBODIMENTS OF THE INVENTION

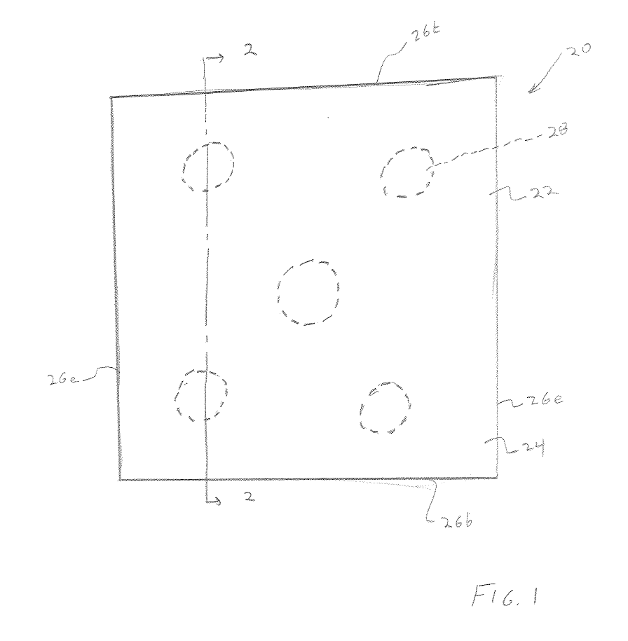

Referring to FIG. 1, a side view of pack 20 according to the invention is shown. The pack 20 has a flexible pouch 22 with a pair of planar sheets 24, only one seen in FIG. 1. The planar sheet 24 has four edges 26, a top edge 26t, a bottom edge 26b, and a pair of side edges 26e. The pack 20 has a plurality of magnets 28, which are shown in hidden line. In the embodiment shown, the pack 20 has five magnets 28.

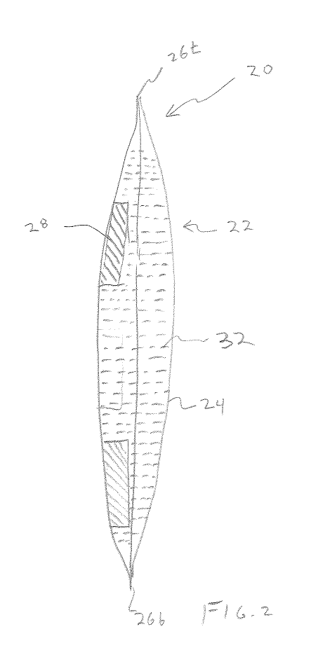

Referring to FIG. 2, a section view of the pack 20 of FIG. 1 is shown. Both of the planar sheets 24 are shown. Two of the magnets 28 are shown in section in the FIG. The pouch 22 of the pack 20 has the pair of planar sheets 24 that are secured together at the four edges 26. In one embodiment, the planar sheets 24 are welded together.

The pouch 22 contains a gel 32. The gel 32 is capable of being cooled and retaining the lack of heat. In an embodiment, the gel 32 is a non-toxic biodegradable gel. In an embodiment, the non-toxic biodegradable gel is p-600 gel ice refrigerant such as marketed and sold by Cold Ice of Oakland, Calif. It is recognized that this particular biodegradable gel 32 can also be heated and retain the heat. In certain embodiments, the material in the pack 20 including the magnets 28, such as hematite, can be microwaved. There are disposable heat and cold packs. It is recognized that the magnets 28 can be placed in disposable packs 20 where two chemicals are mixed by breaking a vial to create a chemical reaction to either heat or cool the pack 20.

In an embodiment, the magnets 28 are permanent magnets. In an embodiment, the permanent magnets 28 are formed of a rare-earth alloy.

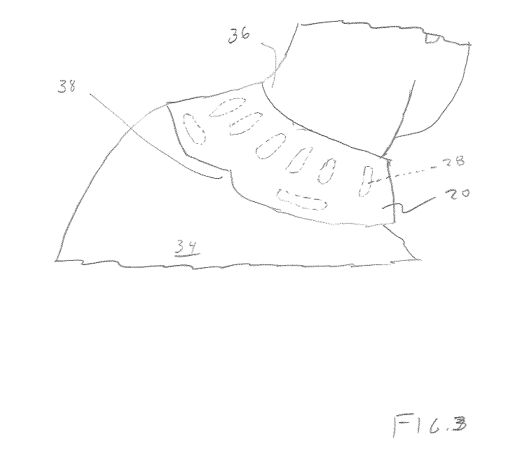

The pack 20 can be heated or cooled prior to applying the pack 20 to the body. While the pack 20 is shown as a square or rectangular in FIG. 1, it is recognized that the pack 20 can be shaped in other forms. Referring to FIG. 3, the pack 20 has a shape to be used around the neck 36 of the user 34. The pack 20 has a shape and is tapered with a notch 38 at the spine to allow the pack to contour to the user's neck. The pack 20 has a plurality of magnets 28 that are positioned to one magnet for each vertebrae in a criss/cross pattern to run the length of the spine, which as we all know is the center of the nervous system.

In a method of therapy of a body including providing a pack 20 having a flexible pouch 22 including a plurality of sheets 24 to form at least one compartment that carries a gel, the pack 20 is placed against the body 34 such that a plurality of magnets 28 carried in the compartment of the pouch work on the body in proximity to the plurality of magnets yielding magnetic therapy. The temperature of the body is adjusted in proximity of the pack by the gel pouch.

The magnets 28 work because the body itself conducts electrical energy. The magnets then arranged in positive to negative sequences help to channel the body's natural energy and thus supplying fresh oxygen and blood to pathways that might otherwise be blocked due to inflammation or to much negative “che”.

The magnets 28 are applied to a specific area of the body such as the neck 36 or back. The magnets 28 influence particles in the blood resulting in the blood moving faster and transfer of more oxygen to the area of the bottom. With more oxygen, the natural healing ability of the person is increased.

Another theory is that the magnets 28 trigger the endorphins which results in the person having a higher pain threshold; the person will not be experiencing pain as long as the magnet is applied to the body. At the same time, the pack 20 is either heating or cooling the area to treat the area.

With respect to the gel 32, the heated pack and the cooled pack serve two different purposes. As indicated below, the treatment of a portion of the body could be with either a heated pack, a cooled pack, or alternating packs.

Generally a use applies treatment for pain or sores. Acute pain is of rapid onset and generally short-lived. Chronic pain, whereas, develops slowly and is persistent and long-lasting.

A heated pack 20 is used to apply heat to the body for pain relief and health. It is sometime used for those with arthritis and stiff muscles and injuries to the deep tissue of the skin. Heat may be an effective self-care treatment for conditions like rheumatoid arthritis. Heat applied superficially to the skin directly increases the temperature of the skin and the tissue immediately underneath the skin.

The therapeutic effects of heat include increasing the extensibility of collagen tissues; decreasing joint stiffness; reducing pain; relieving muscle spasms; reducing inflammation, edema, and aids in the post acute phase of healing; and increasing blood flow. The increased blood flow to the affected area provides proteins, nutrients, and oxygen for better healing. As indicated above with the magnets 28, the magnet also increases blood flow.

As indicated above, the cold therapy with ice is the best immediate treatment for acute injuries because it reduces swelling and pain. Cold therapy is also helpful in treating some overuse injuries or chronic pain in athletes.

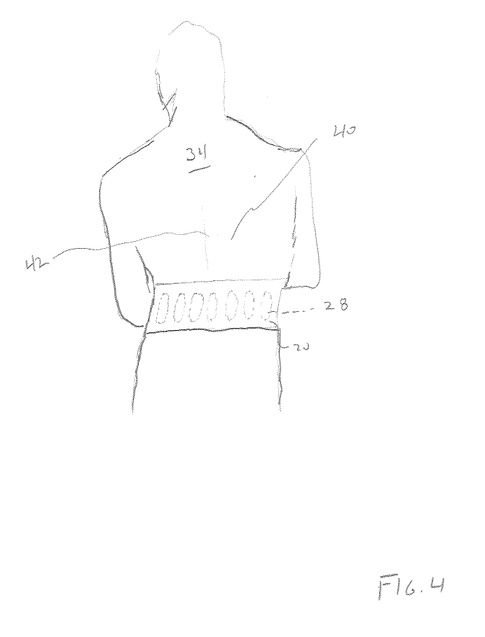

Referring to FIG. 4, a perspective view of an alternative embodiment of a pack 20 on the back 40 of a human 34 is shown. In addition to cooling or heating the back, the pack 20 has the magnet 28 specifically located so that when the pack 20 is placed on the back, the magnets 28 are associated with vertebrae criss/cross positive to negative one for each vertebrae.

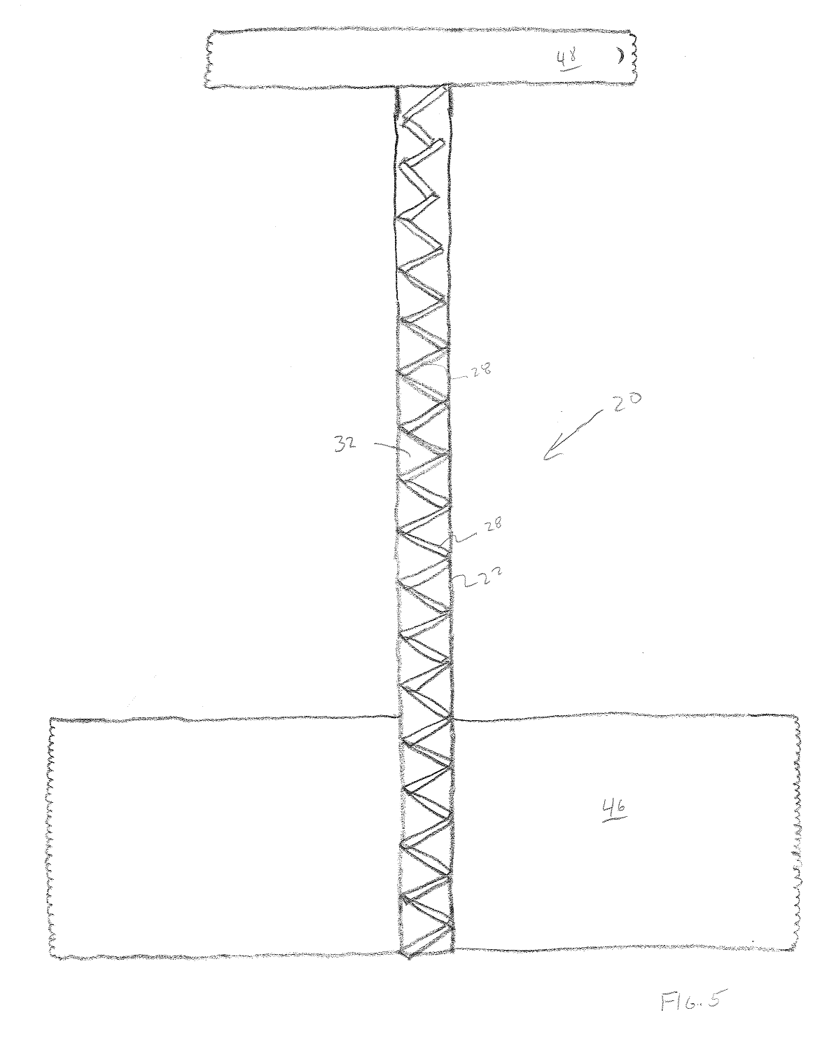

Referring to FIG. 5, a front view of an alternative embodiment of a pack 20 for being placed engagement with the back 40 of a human 34 along the spine 42 of FIG. 4 is shown. The pack 20 has a long narrow flexible pouch 22 which contains the gel 32 and a plurality of magnets 28. The pack 20 has a pair of wraps 46 and 48 for securing the pack 20 to the human body 34. The lower wrap 46 is a wide belt that can go around the waist of human body 34 and has a hook and loop fastener, such as Velcro®, for securing the belt to the body. The upper wrap 48 is narrower and can go around the neck of the human body and be secured with a hook and loop fastener. In the alternative, the upper wrap 48 can be formed similar to the pack 20.

Each of the magnets 28 is long and narrow. The magnets are placed in the pack 20 in a zigzagging pattern so that the end of a magnet 28 is in close proximity to the end of the adjacent magnet 28. When the pack 20 is positioned on the body 34, the pack 20 and the zigzagging pattern of the magnets 28 results in the magnet 28 overlying the spine 42 from the cervical to the coccyx. In a preferred embodiment, there are 32 magnets, one for each vertebrae.

While the principles of the invention have been described herein, it is to be understood by those skilled in the art that this description is made only by way of example and not as a limitation as to the scope of the invention. Other embodiments are contemplated within the scope of the present invention in addition to the exemplary embodiments shown and described herein. Modifications and substitutions by one of ordinary skill in the art are considered to be within the scope of the present invention.

It is recognized that the gel 32 can be two chemicals wherein one of the chemicals is in a vial which is broken in the pouch to mix the chemicals. Depending on the chemicals, the pouch 22 of the pack 20 either rises in temperature or cools relative to ambient temperature.

I claim:

1. A pack comprising:
a flexible pouch having a pair of planar sheets, the sheets each having a top, a bottom, and two opposite side edges, the sheets being secured together along their top, bottom, and side edges to form a compartment; and
a gel carried in the compartment of the pouch, the gel capable of being cooled and retaining the lack of heat; and
a plurality of magnets carried in the compartment of the pouch.

2. A pack of claim 2 wherein the gel is a non-toxic biodegradable gel.

3. A pack of claim 1 wherein the magnets are permanent magnets.

4. A pack of claim 3 wherein the permanent magnets are formed of a rare-earth alloy.

5. A pack comprising:
a flexible pouch having a plurality of sheets to form at least one compartment;
a gel carried in the compartment of the pouch, the gel capable of being varying temperature and retaining the non-ambient temperature for a period of time; and
a plurality of magnets carried in the compartment of the pouch.

6. A pack of claim 5 wherein the gel is capable of being heated and retaining the heat.

7. A pack of claim 5 wherein the gel is capable of being cooled and retaining the lack of heat.

8. A pack of claim 7 wherein the gel is capable of being heated and retaining the heat.

9. A pack of claim 8 wherein the gel is a non-toxic biodegradable gel.

10. A pack of claim 8 wherein the magnets are permanent magnets.

11. A pack of claim 10 wherein the permanent magnets are formed of a rare-earth alloy.

12. A pack of claim 5 wherein the flexible pouch is long and narrow and adapted to align with the spine, each of the magnets orientated to cross across a vertebrae and the pack further comprising a pair of wraps, one at each end of the flexible pouch adapted for securing to the human body.

13. A method of therapy of a body comprising:
providing a pack having a flexible pouch including a plurality of sheets to form at least one compartment that carries a gel;
placing the pack against the body such that a plurality of magnets carried in the compartment of the pouch such that magnetic therapy works on the body in proximity to the plurality of magnets; and
adjusting temperature of the body in proximity of the pack by the gel pouch.

14. A method of claim 13 wherein the pack including the gel is heated before applying to body.

15. A method of claim 13 wherein the pack including the gel is cooled before applying to body.

16. A method of claim 15 wherein the pack is designed for and applied to the neck.

17. A method of claim 15 wherein the pack is designed for and applied to the lower back.

18. A method of claim 15 wherein the pack is designed for and applied to the back to align with the spine.

19. A method of claim 18 wherein the pack further comprises a pair of wraps, one wrap at each end of the flexible pouch adapted for securing the pack to the human body.
